(19) United States
(12) Patent Application Publication
(10) Pub. No.: US 2026/0364342 A1
(43) Pub. Date: Nov. 19, 2026

(54) APPARATUS AND METHOD FOR SUPPLYING CURRENT OF MONOPOLAR AND BIPOLAR BY SKIN DEPTH FOR PERFORMING REPEATED NEEDLE INSERTION MOVEMENTS

(71) Applicant: SSB MEDICAL COOPERATION, Seoul (KR)

(72) Inventors: Suk Bae SEO, Seoul (KR); Ja Young KIM, Seoul (KR); Ho Joon SEO, Seoul (KR)

(73) Assignee: SSB MEDICAL COOPERATION, Seoul (KR)

(21) Appl. No.: 19/669,019

(22) Filed: May 6, 2026

Related U.S. Application Data
(63) Continuation of application No. PCT/KR2024/017200, filed on Nov. 4, 2024.

(30) Foreign Application Priority Data
Nov. 7, 2023 (KR) 10-2023-0152401

(51) Int. Cl. A61N 1/06; A61N 1/05

(52) U.S. Cl. CPC A61N 1/06; A61N 1/0502

(57) ABSTRACT

The present disclosure may include an electrode unit including a plurality of electrodes of a needle type that repeatedly performs an injection operation and a pull operation within a skin, a drug injection unit that injects a drug into the skin, an energy supply unit that selectively applies a first high-frequency current of a monopolar type and a second high-frequency current of a bipolar type to the plurality of electrodes, a transfer unit that moves the electrode unit such that the plurality of electrodes reach different target depths within the skin by repeatedly performing the injection operation and the pull operation at a same location, and a processor that, when the plurality of electrodes respectively reach different target depths by repeatedly performing the injection operation and the pull operation, controls the energy supply unit such that the first high-frequency current and the second high-frequency current are applied to the plurality of electrodes at current intensities corresponding to the reached different target depths, respectively, and controls the drug injection unit such that the drug is injected into the different target depths.

### CROSS-REFERENCE TO RELATED APPLICATIONS

The present application is a continuation of International Patent Application No. PCT/KR2024/017200, filed on Nov. 4, 2024, which is based upon and claims the benefit of priority to Korean Patent Application No. 10-2023-0152401 filed on Nov. 7, 2023. The disclosures of the above-listed applications are hereby incorporated by reference herein in their entirety.

### TECHNICAL FIELD

The present disclosure relates to an apparatus and a method for applying monopolar and bipolar currents. More specifically, the present disclosure relates to an apparatus and a method for applying monopolar and bipolar currents at different skin depths for performing a repetitive needle injection operation.

### BACKGROUND TECHNOLOGY

In general, skin care apparatuses for removing wrinkles, restoring skin elasticity, and removing sebum use a method (HIFU type) of delivering ultrasound to skin tissues, a method (RF type) of delivering high-frequency waves to skin tissues, and a method (Optical type) of irradiating laser light to skin tissues.

The apparatus that delivers high-frequency waves to skin tissues may repeatedly invade a deep portion (e.g., a dermis layer) of the skin (e.g., a face) with an RF needle electrode moving back and forth in a vertical direction, may remove damaged collagen, elastic fibers, or the like from the deep portion of the skin at a target point by using the heat generated by high-frequency waves, and may promote new formation.

Furthermore, the skin care apparatus improves skin pigmentation, acne scars, and wrinkles.

In other words, after a variety of energy is applied to a target area of the skin to intentionally cause wounds, the target area of the skin is regenerated by stimulating collagen in the dermis layer and inducing collagen regeneration.

However, conventional skin care apparatuses are incapable of delivering abundant or efficient electrical energy to specific depths of a skin.

Accordingly, the conventional skin care apparatuses may not accurately deliver abundant or efficient electrical energy during ultrasonic irradiation, thereby resulting in reduced accuracy of the ultrasonic irradiation.

Moreover, since doctors need to carefully perform ultrasound treatments, the conventional skin care apparatuses have limitations in shortening treatment time and maximizing the effectiveness of ultrasound treatments.

### DETAILED DESCRIPTION OF INVENTION

### Technical Problem

The purpose of an embodiment disclosed in the present disclosure is to provide a method of increasing the accuracy of ultrasonic irradiation by accurately irradiating abundant or efficient electrical energy during ultrasonic irradiation.

Moreover, the purpose of an embodiment disclosed in the present disclosure is to provide a method of shortening the ultrasonic irradiation time and maximizing the effect of the ultrasonic irradiation.

Furthermore, the purpose of an embodiment disclosed in the present disclosure is to provide a method of maximizing the optimal skin improvement effect of both monopolar and bipolar types while preventing the occurrence of skin burns.

Besides, the purpose of an embodiment disclosed in the present disclosure is to provide a method of reducing pain caused by needle injection by implementing a needle in a manner of gradually increasing the depth.

Besides, the purpose of an embodiment disclosed in the present disclosure is to provide a method of injecting a drug into a gradually deeper location.

Problems to be solved by the present disclosure are not limited to the problems mentioned above, and other problems not mentioned will be apparent by those skilled in the art from the following description.

### Technical Solution

According to one aspect of the present disclosure for achieving the aforementioned technical problem, a skin-depth-specific monopolar and bipolar current application apparatus for performing a repetitive needle injection operation may include an electrode unit including a plurality of electrodes of a needle type that repeatedly performs an injection operation and a pull operation within a skin, a drug injection unit that injects a drug into the skin, an energy supply unit that selectively applies a first high-frequency current of a monopolar type and a second high-frequency current of a bipolar type to the plurality of electrodes, a transfer unit that moves the electrode unit such that the plurality of electrodes reach different target depths within the skin by repeatedly performing the injection operation and the pull operation at a same location, and a processor that, when the plurality of electrodes respectively reach the different target depths by repeatedly performing the injection operation and the pull operation, controls the energy supply unit such that the first high-frequency current and the second high-frequency current are applied to the plurality of electrodes at current intensities corresponding to the reached different target depths, respectively, and controls the drug injection unit such that the drug is injected into the different target depths.

Moreover, the processor may control the energy supply unit such that the first high-frequency current is applied to the plurality of electrodes at a current intensity corresponding to the first depth, when the plurality of electrodes perform the injection operation for a first time, and the target depth is a predetermined first depth, and may control the energy supply unit such that the second high-frequency current is applied to the plurality of electrodes at a current intensity corresponding to the second depth when the plurality of electrodes perform the injection operation for a second time, and the target depth is a predetermined second depth deeper than the first depth.

Furthermore, the processor may control the energy supply unit such that the first high-frequency current is applied to the plurality of electrodes at a current intensity corresponding to the third depth when the plurality of electrodes perform the injection operation for a third time, and the target depth is a predetermined third depth deeper than the second depth.

Besides, the processor may control the energy supply unit such that the second high-frequency current is applied to the plurality of electrodes at a current intensity corresponding to the first depth when the plurality of electrodes perform the injection operation for a first time, and the target depth is a predetermined first depth, and may control the energy supply unit such that the first high-frequency current is applied to the plurality of electrodes at a current intensity corresponding to the second depth when the plurality of electrodes perform the injection operation for a second time, and the target depth is a predetermined second depth deeper than the first depth.

Also, the processor may control the energy supply unit such that the second high-frequency current is applied to the plurality of electrodes at a current intensity corresponding to the third depth when the plurality of electrodes perform the injection operation for a third time, and the target depth is a predetermined third depth deeper than the second depth.

Moreover, according to one aspect of the present disclosure for achieving the aforementioned technical problem, a method performed by a skin-depth-specific monopolar and bipolar current application apparatus for performing a repetitive needle injection operation may include moving an electrode unit of the apparatus such that a plurality of electrodes reach different target depths within a skin through a transfer unit of the apparatus by repeatedly performing an injection operation and a pull operation at a same location, determining, by a processor of the apparatus, whether the plurality of electrodes respectively reach different target depths through repeated performance of the injection operation and the pull operation, when the plurality of electrodes respectively reach different target depths by repeatedly performing the injection operation and the pull operation, controlling, by the processor, an energy supply unit of the apparatus such that the first high-frequency current and the second high-frequency current are applied to the plurality of electrodes respectively at current intensities respectively corresponding to the reached different target depths, and controlling a drug injection unit of the apparatus such that the drug is injected into the different target depths.

Moreover, the controlling may include controlling, by the processor, the energy supply unit such that the first high-frequency current is applied to the plurality of electrodes at a current intensity corresponding to the first depth when the plurality of electrodes perform the injection operation for a first time, and the target depth is a predetermined first depth, and controlling, by the processor, the energy supply unit such that the second high-frequency current is applied to the plurality of electrodes at a current intensity corresponding to the second depth when the plurality of electrodes perform the injection operation for a second time, and the target depth is a predetermined second depth deeper than the first depth.

Furthermore, the controlling may include controlling, by the processor, the energy supply unit such that the first high-frequency current is applied to the plurality of electrodes at a current intensity corresponding to the third depth when the plurality of electrodes perform the injection operation for a third time, and the target depth is a predetermined third depth deeper than the second depth.

Besides, the controlling may include controlling, by the processor, the energy supply unit such that the second high-frequency current is applied to the plurality of electrodes at a current intensity corresponding to the first depth when the plurality of electrodes perform the injection operation for a first time, and the target depth is a predetermined first depth, and controlling, by the processor, the energy supply unit such that the first high-frequency current is applied to the plurality of electrodes at a current intensity corresponding to the second depth when the plurality of electrodes perform the injection operation for a second time, and the target depth is a predetermined second depth deeper than the first depth.

Also, the controlling may include controlling, by the processor, the energy supply unit such that the second high-frequency current is applied to the plurality of electrodes at a current intensity corresponding to the third depth when the plurality of electrodes perform the injection operation for a third time, and the target depth is a predetermined third depth deeper than the second depth.

### Effect of Invention

According to the aforementioned problem-solving means of the present disclosure, it is possible to increase the accuracy of ultrasonic irradiation by accurately irradiating abundant or efficient electrical energy during ultrasonic irradiation.

Moreover, according to the aforementioned problem-solving means of the present disclosure, it is possible to shorten the ultrasonic irradiation time and to maximize the effect of the ultrasonic irradiation.

Furthermore, according to the aforementioned problem-solving means of the present disclosure, it is possible to maximize the optimal skin improvement effect of both monopolar and bipolar types while it is possible to prevent the occurrence of skin burns.

Besides, according to the aforementioned problem-solving means of the present disclosure, it is possible to reduce pain caused by needle injection by implementing a needle in a manner of gradually increasing the depth.

Also, according to the aforementioned problem-solving means of the present disclosure, it is possible to inject a drug into a gradually deeper location.

Effects of the present disclosure are not limited to the effects mentioned above, and other effects not mentioned will be apparent by those skilled in the art from the following description.

### DESCRIPTION OF DRAWINGS

FIG. 1 is a diagram showing a configuration of a skin-depth-specific monopolar and bipolar current application apparatus for performing repetitive needle injection operations, according to an embodiment of the present disclosure.

FIG. 2 is a diagram illustrating a process, in which a plurality of electrodes repeatedly perform injection and pull operations at the same location by moving a transfer unit of FIG. 1, as an example.

FIG. 3 is a diagram illustrating a structure of a plurality of needle-type electrodes placed in an electrode unit of FIG. 1.

FIG. 4 is a diagram illustrating a process of applying a first high-frequency current of a monopolar type to a plurality of needle-type electrodes of FIG. 3, as an example.

FIG. 5 is a diagram illustrating a process of applying a second high-frequency current of a bipolar type to a plurality of needle-type electrodes of FIG. 3, as an example.

FIG. 6 is a flowchart illustrating a skin-depth-specific monopolar and bipolar current application method for performing repetitive needle injection operations, according to an embodiment of the present disclosure.

FIGS. 7 to 10 are diagrams illustrating a process of allowing a first high-frequency current, a second high-frequency current, and the first high-frequency current to be applied to a plurality of electrodes through each injection operation in the control step of FIG. 6, as an example.

FIGS. 11 to 14 are diagrams illustrating a process of allowing a second high-frequency current, a first high-frequency current, and the second high-frequency current to be applied to a plurality of electrodes through each injection operation in the control step of FIG. 6, as an example.

### FORMS OF IMPLEMENTATION OF INVENTION

The same reference numerals denote the same elements throughout the present disclosure. The present disclosure does not describe all elements of embodiments. Well-known content in a technical field, to which the present disclosure belongs, or redundant content in which embodiments are the same as one another will be omitted. A term such as ‘unit, module, member, or block’ used in the specification may be implemented with software or hardware. According to embodiments, a plurality of ‘units, modules, members, or blocks’ may be implemented with one component, or a single ‘unit, module, member, or block’ may include a plurality of components.

Throughout this specification, when it is supposed that a portion is “connected” to another portion, this includes not only a direct connection, but also an indirect connection. The indirect connection includes being connected through a wireless communication network.

Furthermore, when a portion “comprises” a component, it will be understood that it may further include another component, without excluding other components unless specifically stated otherwise.

Throughout this specification, when it is supposed that a member is located on another member “on”, this includes not only the case where one member is in contact with another member but also the case where another member is present between two other members.

Terms such as ‘first’, ‘second’, and the like are used to distinguish one component from another component, and thus the component is not limited by the terms described above.

Unless there are obvious exceptions in the context, a singular form includes a plural form.

In each step, an identification code is used for convenience of description. The identification code does not describe the order of each step. Unless the context clearly states a specific order, each step may be performed differently from the specified order.

Hereinafter, operating principles and embodiments of the present disclosure will be described with reference to the accompanying drawings.

According to an embodiment of the present disclosure, a skin-depth-specific monopolar and bipolar current application apparatus during needle injection is used to raise the temperature of the electrically conductive target area beneath a skin surface via microneedles to a level at which tissue heating occurs.

In this specification, a control unit of the skin-depth-specific monopolar and bipolar current application apparatus according to an embodiment of the present disclosure that performs repeated needle injection operations includes all various devices capable of providing results to a user by performing arithmetic processing. For example, the control unit of the skin-depth-specific monopolar and bipolar current application apparatus according to an embodiment of the present disclosure that performs repeated needle injection operations may include all of a computer, a server device, and a portable terminal, or may take the form of any one of these.

Here, for example, the computer may include a notebook computer, a desktop computer, a laptop computer, a tablet PC, a slate PC, and the like, which are equipped with a web browser.

A server device may be a server that processes information by communicating with an external device and may include an application server, a computing server, a database server, a file server, a mail server, a proxy server, and a web server.

For example, the portable terminal may be a wireless communication device that guarantees portability and mobility, and may include all kinds of handheld-based wireless communication devices such as a smartphone, a personal communication system (PCS), a global system for mobile communication (GSM), a personal digital cellular (PDC), a personal handyphone system (PHS), a personal digital assistant (PDA), International Mobile Telecommunication (IMT)-2000, a code division multiple access (CDMA)-2000, W-Code Division Multiple Access (W-CDMA), and Wireless Broadband Internet terminal (Wibro) terminal, and a wearable device such as a timepiece, a ring, a bracelet, an anklet, a necklace, glasses, a contact lens, or a head-mounted device (HMD).

A skin-depth-specific monopolar and bipolar current application apparatus for performing a repetitive needle injection operation according to an embodiment of the present disclosure may control an energy supply unit such that the first high-frequency current of a monopolar type and the second high-frequency current of a bipolar type are applied to the plurality of electrodes at current intensities corresponding to the reached different target depths, respectively, and may control a drug injection unit such that the drug is injected into the different target depths, when a plurality of electrodes respectively reach different target depths by repeatedly performing the injection operation and the pull operation. In this case, the intensity of the first monopolar-type high-frequency current and the second bipolar-type high-frequency current increases as the target depth increases, but their intensities may differ from each other.

Hereafter, the skin-depth-specific monopolar and bipolar current application apparatus for performing repetitive needle injection operations will be described in detail.

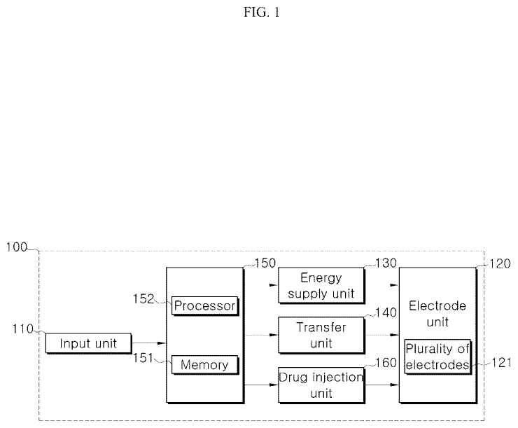

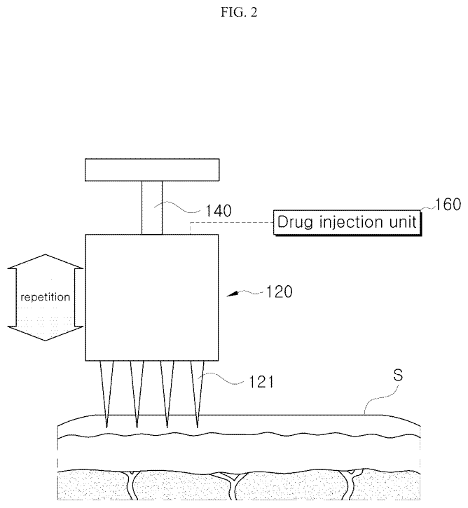

FIG. 1 is a diagram showing a configuration of a skin-depth-specific monopolar and bipolar current application apparatus for performing repetitive needle injection operations, according to an embodiment of the present disclosure. FIG. 2 is a diagram illustrating a process, in which a plurality of electrodes repeatedly perform injection and pull operations at the same location by moving a transfer unit of FIG. 1, as an example.

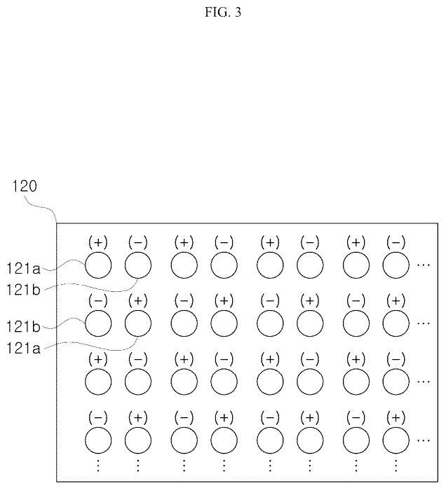

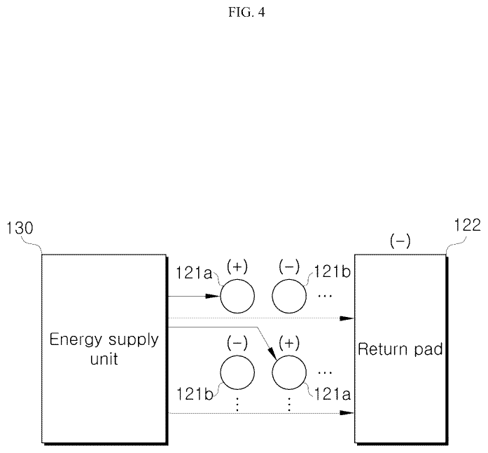

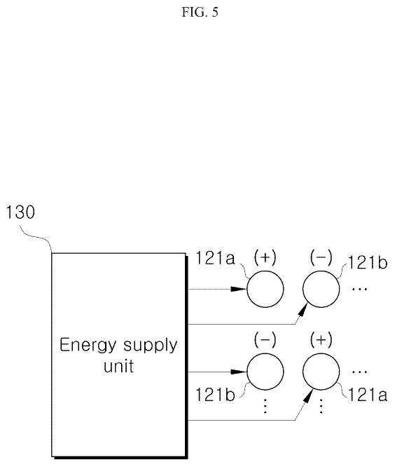

FIG. 3 is a diagram illustrating a structure of a plurality of needle-type electrodes placed in an electrode unit of FIG. 1. FIG. 4 is a diagram illustrating a process of applying a first high-frequency current of a monopolar type to a plurality of needle-type electrodes of FIG. 3, as an example. FIG. 5 is a diagram illustrating a process of applying a second high-frequency current of a bipolar type to a plurality of needle-type electrodes of FIG. 3, as an example.

Referring to FIGS. 1 to 5, a skin-depth-specific monopolar and bipolar current application apparatus 100 that performs a needle repetitive injection operation may include an input unit 110, an electrode unit 120, an energy supply unit 130, a transfer unit 140, and a control unit 150.

The input unit 110 is used to receive target depth information within a skin from a user. When the target depth information within the skin is received, the control unit 150 may control the operation of the present apparatus to correspond to the received target depth information within the skin. Here, the target depth information within the skin may be a target depth value within a dermis layer.

The input unit 110 may include a hardware-type physical key (e.g., a button, a dome switch, a jog wheel, or a jog switch that is located on at least one of the front, back, and sides of the present apparatus) and a software-type touch key. For example, the touch key may consist of a virtual key, a soft key, or a visual key displayed on a touch screen-type display unit through software processing or may consist of a touch key positioned on a portion other than a touch screen. In the meantime, the virtual key or the visual key may be displayed on the touch screen while having various shapes. For example, the virtual key or visual key may be formed of graphics, texts, icons, video, or a combination thereof.

The electrode unit 120 may include a plurality of electrodes 121 of a needle type that repeatedly perform injection and pull operations within a skin ‘S’. The plurality of electrodes 121 of a needle type penetrate the deep part (e.g., a dermis layer) of the skin ‘S’ through repeated injection operations, remove damaged collagen, elastic fibers, etc., from the deep part of the target point by using heat generated by high frequency, and promote new formation.

As shown in FIG. 3, among the plurality of electrodes 121 of the needle type, a plurality of electrodes 121a of (+) polarity and a plurality of electrodes 121b of (−) polarity may be placed in a plurality of group forms, and the plurality of electrodes 121b of (−) polarity and the plurality of electrodes 121a of (+) polarity may be placed in a plurality of group forms. For example, the plurality of electrodes 121a of (+) polarity and the plurality of electrodes 121b of (−) polarity, which are in the plurality of group forms, may be repeatedly placed in the odd-numbered rows of the electrode unit 120, and the plurality of electrodes 121b of (−) polarity and the plurality of electrodes 121a of (+) polarity, which are in the plurality of group forms, may be repeatedly placed in the even-numbered rows of the electrode unit 120.

The energy supply unit 130 may selectively apply the first high-frequency current of a monopolar type and the second high-frequency current of a bipolar type to the plurality of electrodes 121. Here, the energy supply unit 130 may include a switching circuit for selectively applying the first high-frequency current of a monopolar type and the second high-frequency current of a bipolar type.

In this case, as illustrated in FIG. 4, the energy supply unit 130 may apply the first high-frequency current of a monopolar type to the plurality of electrodes 121a of (+) polarity and a return pad 122 of (−) polarity from among the plurality of electrodes 121. A current circuit needs to be formed such that a high-frequency current flows through the skin ‘S’. When the energy supply unit 130 applies the first high-frequency current of a monopolar type to the plurality of electrodes 121a of (+) polarity and the return pad 122 of (−) polarity, high-frequency energy is concentrated on the multiple electrodes 121a of the (+) polarity, thereby enabling deep heat transfer compared to the bipolar type to generate abundant heat in the deep tissue. Here, the return pad 122 may be a pad for grounding.

Moreover, as shown in FIG. 5, the energy supply unit 130 may apply the second high-frequency current of a bipolar type to the plurality of electrodes 121a of (+) polarity and the plurality of electrodes 121b of (−) polarity from among the plurality of electrodes 121. When the energy supply unit 130 applies the second high-frequency current of a bipolar type to the plurality of electrodes 121a of (+) polarity and the plurality of electrodes 121b of (−) polarity, a current circuit may be formed between the plurality of electrodes 121a of (+) polarity and the plurality of electrodes 121b of (−) polarity, thereby preventing excessive heat transfer and allowing the high-frequency irradiation to be concentrated on the selected area.

The transfer unit 140 may move the electrode unit 120 such that the plurality of electrodes 121 reach different target depths within the skin by repeatedly performing injection and pull operations at the same location. In this case, the transfer unit 140 may move the electrode unit 120 in a vertical direction.

The control unit 150 may be implemented with a memory 151 that stores data regarding an algorithm for controlling operations of components within the present device, or a program for realizing the algorithm, and at least one processor 152 that performs the above-described operation by using the data stored in the memory 151. Here, each of the memory 151 and the processor 152 may be implemented as separate chips. Moreover, the memory 151 and the processor 152 may be implemented as a single chip.

The memory 151 may store data that supports various functions of the present apparatus, and a program for operations of the control unit, may store input/output data, and may store a plurality of application programs (or applications) running on the present apparatus, pieces of data for operations of the present apparatus, and instructions. At least part of the application programs may be downloaded from an external server through wireless communication.

The memory 151 may include the type of a storage medium of at least one of a flash memory type, a hard disk type, a solid state disk (SSD) type, a silicon disk drive (SDD) type, a multimedia card micro type, a memory of a card type (e.g., SD memory, XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disc. Furthermore, the memory 151 may be separate from the present device, but may be a database connected by wire or wirelessly.

The memory 151 may store depth information about a skin layer and data related to the repetitive needle injection operation. The processor 152 may control an operation related to the repetitive needle injection operation.

When the plurality of electrodes 121 reach different target depths of a skin layer through the repeated performance of injection and pull operations, the processor 152 may control the energy supply unit 130 such that the first high-frequency current of a monopolar type and the second high-frequency current of a bipolar type are applied to the plurality of electrodes 121 at current intensities corresponding to the different target depths thus reached. In this case, the intensity of the first monopolar-type high-frequency current and the second bipolar-type high-frequency current increases as the target depth increases, but their intensities may differ from each other. For example, as the target depth increases, the intensity of the first high-frequency current may be greater or smaller than the intensity of the second high-frequency current.

The processor 152 may control the energy supply unit 130 such that when the plurality of electrodes 121 perform an injection operation for the first time and the target depth is a predetermined first depth, the first high-frequency current is applied to the plurality of electrodes 121 with a current intensity corresponding to the first depth. Moreover, the processor 152 may control the energy supply unit 130 such that when the plurality of electrodes 121 perform an injection operation for the second time and the target depth is a predetermined second depth deeper than the first depth, the second high-frequency current is applied to the plurality of electrodes 121 with a current intensity corresponding to the second depth. The processor 152 may control the energy supply unit 130 such that when the plurality of electrodes 121 perform an injection operation for the third time and the target depth is a predetermined third depth deeper than the second depth, the first high-frequency current is applied to the plurality of electrodes 121 with a current intensity corresponding to the third depth.

The processor 152 may control the energy supply unit 130 such that when the plurality of electrodes 121 perform an injection operation for the first time and the target depth is a predetermined first depth, the second high-frequency current is applied to the plurality of electrodes 121 with a current intensity corresponding to the first depth. Furthermore, the processor 152 may control the energy supply unit 130 such that when the plurality of electrodes 121 perform an injection operation for the second time and the target depth is the predetermined second depth deeper than the first depth, the first high-frequency current is applied to the plurality of electrodes 121 with a current intensity corresponding to the second depth. The processor 152 may control the energy supply unit 130 such that when the plurality of electrodes 121 perform an injection operation for the third time and the target depth is a predetermined third depth deeper than the second depth, the second high-frequency current is applied to the plurality of electrodes 121 with a current intensity corresponding to the third depth.

The processor 152 may control a drug injection unit 160 such that when the plurality of electrodes 121 reach different target depths of the skin layer through the repeated performance of injection and pull operations, a drug for skin care is injected into different target depths of the skin layer.

Meanwhile, the processor 152 may control a display device to monitor the plurality of electrodes 121 that are injected at each injection depth whenever the plurality of electrodes 121 proceed at each injection depth of the skin layer. Besides, whenever the plurality of electrodes 121 proceed at each injection depth of the skin layer, the processor 152 may measure the impedance for each current at each injection depth and may adjust and correct energy at each injection depth based on the measured impedance.

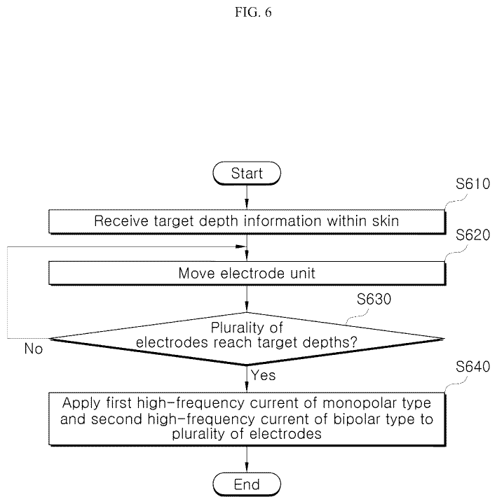

FIG. 6 is a flowchart illustrating a skin-depth-specific monopolar and bipolar current application method for performing repetitive needle injection operations, according to an embodiment of the present disclosure.

Referring to FIG. 6, a skin-depth-specific monopolar and bipolar current application method for performing a repetitive needle injection operation may include input step S610, movement step S620, determination step S630, and control step S640.

The input unit 110 may receive target depth information within a skin from a user (S610). Here, the target depth information within the skin may be a target depth value within a dermis layer (S610).

The transfer unit 140 may move the electrode unit 120 such that the plurality of electrodes 121 reach different target depths within the skin ‘S’ by repeatedly performing injection and pull operations at the same location (S620). The processor 152 may control the transfer unit 140, which moves the electrode unit 120 such that the plurality of electrodes 121 reach different target depths within the skin ‘S’ by repeatedly performing injection and pull operations at the same location based on target depth information within the skin ‘S’ input through the input unit 110. In this case, the plurality of electrodes 121 may penetrate to their respective target depths within a dermis layer.

The processor 152 may determine whether the plurality of electrodes 121 respectively reach different target depths through the repeated performance of injection and pull operations (S630). For example, the processor 152 may determine whether the plurality of electrodes 121 reach their respective target depths within the dermis layer.

When the plurality of electrodes 121 reach different target depths through the repeated performance of injection and pull operations (S630), the processor 152 may control the energy supply unit 130 such that a first high-frequency current of a monopolar type and a second high-frequency current of a bipolar type are respectively applied to the plurality of electrodes 121 at current intensities corresponding to the reached different target depths, and may control the drug injection unit 160 such that a drug for skin care is injected into the different target depths (S640). Here, the energy supply unit 130 may selectively apply the first high-frequency current of a monopolar type and the second high-frequency current of a bipolar type to the plurality of electrodes 121.

In this case, the intensity of the first monopolar-type high-frequency current and the second bipolar-type high-frequency current increases as the target depth increases, but their intensities may differ from each other. Moreover, the drug injection unit 160 may inject a drug for skin care into each target depth within the dermis layer. For example, as the target depth increases, the intensity of the first high-frequency current of the monopolar type may be greater than the intensity of the second high-frequency current of the bipolar type. That is, throughout the entire time that the plurality of electrodes 121 reach the target depth within the dermis layer, the intensities of the first high-frequency current of the monopolar type may be greater than those of the second high-frequency current of the bipolar type. For another example, as the target depth increases, the intensity of the second high-frequency current of the bipolar type may be greater than the intensity of the first high-frequency current of the monopolar type. That is, throughout the entire time that the plurality of electrodes 121 reach the target depth within the dermis layer, the intensities of the second high-frequency current of the bipolar type may be greater than those of the first high-frequency current of the monopolar type.

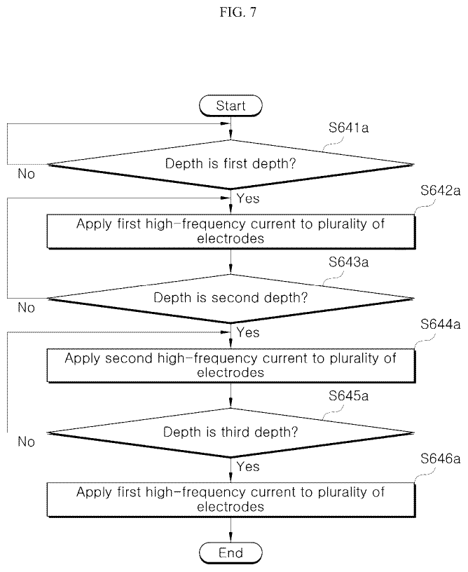

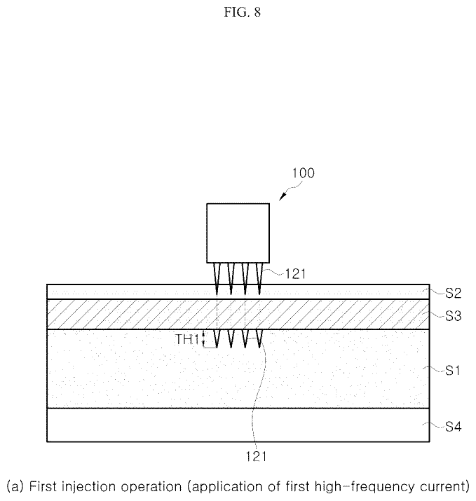

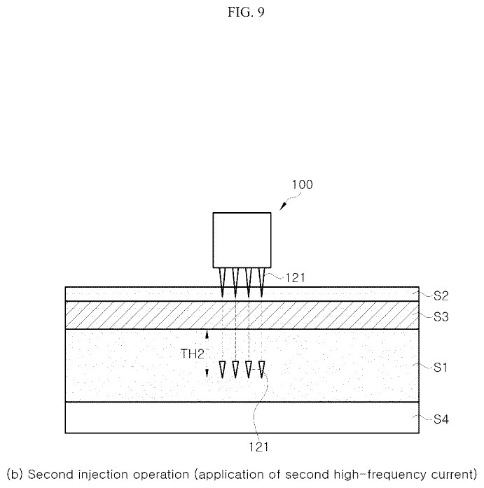

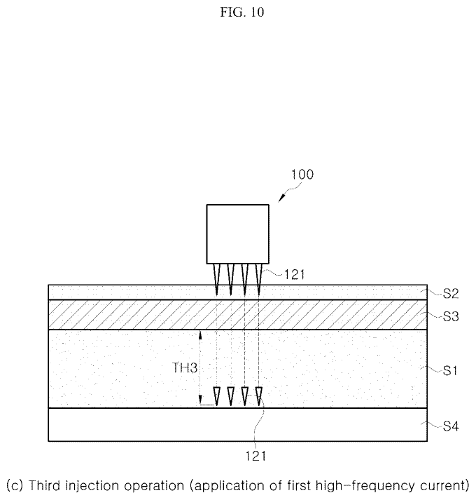

FIGS. 7 to 10 are diagrams illustrating a process of allowing a first high-frequency current, a second high-frequency current, and the first high-frequency current to be applied to a plurality of electrodes through each injection operation in the control step of FIG. 6, as an example.

Referring to FIGS. 7 to 10, the processor 152 may determine whether the plurality of electrodes 121 perform the injection operation for the first time and the target depth is a predetermined first depth TH1 (S641a). When the target depth within a dermis layer S1 is the predetermined first depth TH1 (S641a), the processor 152 may control the energy supply unit 130 such that the first high-frequency current of a monopolar type is applied to the plurality of electrodes 121 with a current intensity corresponding to the first depth TH1 (S642a). In this case, S2 may be a stratum corneum; S3 may be an epidermis layer; and, S4 may be a subcutaneous tissue layer. Moreover, the processor 152 may allow the drug injection unit 160 to inject a drug for skin care into the first depth TH1 within the dermis layer (S642a).

The processor 152 may determine whether the plurality of electrodes 121 perform an injection operation for the second time and the target depth is a second depth TH2 deeper than the predetermined first depth TH1 (S643a). When the target depth within the dermis layer S1 is the predetermined second depth TH2 (S643a), the processor 152 may control the energy supply unit 130 such that the second high-frequency current of a bipolar type is applied to the plurality of electrodes 121 with a current intensity corresponding to the second depth TH2 (S644a). Furthermore, the processor 152 may allow the drug injection unit 160 to inject a drug for skin care into the second depth TH2 within the dermis layer (S644a). In this case, the amount of drug injected at the second depth TH2 may be less or more than the amount of drug injected at the first depth TH1.

Meanwhile, when the plurality of electrodes 121 perform an injection operation for the second time and the target depth reaches the predetermined second depth TH2, the processor 152 may allow the plurality of electrodes 121 to have a predetermined first rest period between application time points of the first high-frequency current of a monopolar type and the second high-frequency current of a bipolar type. Here, the first rest period may be a period during which the second high-frequency current of a bipolar type is not applied during a predetermined time. The present disclosure may have the first rest period, thereby preventing the occurrence of skin burns in advance and maximizing the optimal skin improvement effect of the monopolar and bipolar types.

The processor 152 may determine whether the plurality of electrodes 121 perform an injection operation for the third time and the target depth is a third depth TH3 deeper than the predetermined second depth TH2 (S645a). When the target depth within the dermis layer S1 is the predetermined third depth TH3 (S645a), the processor 152 may control the energy supply unit 130 such that the first high-frequency current of a monopolar type is applied to the plurality of electrodes 121 with a current intensity corresponding to the third depth TH3 (S646a). In this case, the amount of drug injected at the third depth TH3 may be less or more than the amount of drug injected at the second depth TH2.

Meanwhile, when the plurality of electrodes 121 perform an injection operation for the third time and the target depth reaches the predetermined third depth TH3, the processor 152 may allow the plurality of electrodes 121 to have a predetermined second rest period between application time points of the second high-frequency current of a bipolar type and the first high-frequency current of a monopolar type. Here, the second rest period may be a period during which the first high-frequency current of a monopolar type is not applied during a predetermined time. The present disclosure may have the second rest period, thereby preventing the occurrence of skin burns in advance and maximizing the optimal skin improvement effect of the monopolar and bipolar types.

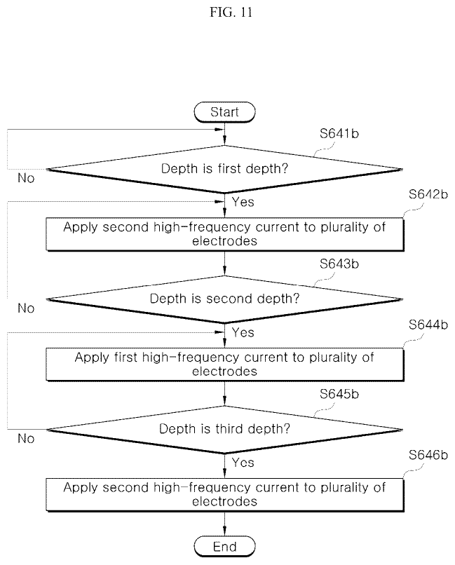

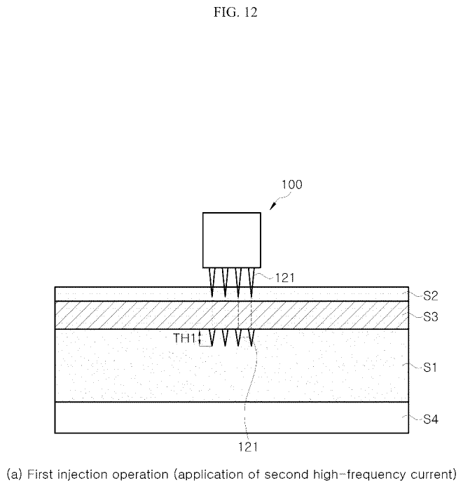

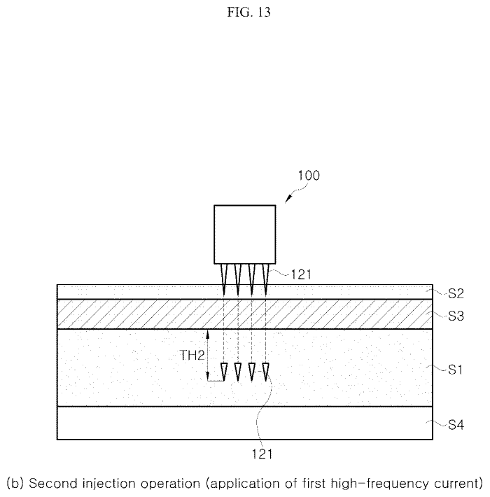

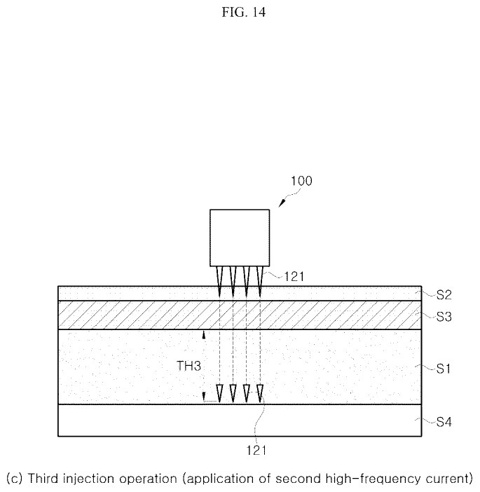

FIGS. 11 to 14 are diagrams illustrating a process of allowing a second high-frequency current, a first high-frequency current, and the second high-frequency current to be applied to a plurality of electrodes through each injection operation in the control step of FIG. 6, as an example.

Referring to FIGS. 11 to 14, the processor 152 may determine whether the plurality of electrodes 121 perform the injection operation for the first time and the target depth is a predetermined first depth TH1 (S641b). When the target depth within a dermis layer S1 is the predetermined first depth TH1 (S641b), the processor 152 may control the energy supply unit 130 such that the second high-frequency current of a bipolar type is applied to the plurality of electrodes 121 with a current intensity corresponding to the first depth TH1 (S642a). In this case, S2 may be a stratum corneum; S3 may be an epidermis layer; and, S4 may be a subcutaneous tissue layer. Moreover, control step S642b may allow the drug injection unit 160 to inject a drug for skin care into the first depth TH1 within the dermis layer through the processor 152.

The processor 152 may determine whether the plurality of electrodes 121 perform an injection operation for the second time and the target depth is a second depth TH2 deeper than the predetermined first depth TH1 (S643b). When the target depth within the dermis layer S1 is the predetermined second depth TH2 (S643b), the processor 152 may control the energy supply unit 130 such that the first high-frequency current of a monopolar type is applied to the plurality of electrodes 121 with a current intensity corresponding to the second depth TH2 (S644a). Furthermore, the processor 152 may allow the drug injection unit 160 to inject a drug for skin care into the second depth TH2 within the dermis layer (S644b). In this case, the amount of drug injected at the second depth TH2 may be less or more than the amount of drug injected at the first depth TH1.

Meanwhile, when the plurality of electrodes 121 perform an injection operation for the second time and the target depth reaches the predetermined second depth TH2, the processor 152 may allow the plurality of electrodes 121 to have a predetermined third rest period between application time points of the second high-frequency current of a bipolar type and the first high-frequency current of a monopolar type. Here, the third rest period may be a period during which the first high-frequency current of a monopolar type is not applied during a predetermined time. The present disclosure may have the third rest period, thereby preventing the occurrence of skin burns in advance and maximizing the optimal skin improvement effect of the monopolar and bipolar types.

The processor 152 may determine whether the plurality of electrodes 121 perform an injection operation for the third time and the target depth is a third depth TH3 deeper than the predetermined second depth TH2 (S645b). When the target depth within the dermis layer S1 is the predetermined third depth TH3 (S645b), the processor 152 may control the energy supply unit 130 such that the second high-frequency current of a bipolar type is applied to the plurality of electrodes 121 with a current intensity corresponding to the third depth TH3 (S646b). In this case, the amount of drug injected at the third depth TH3 may be less or more than the amount of drug injected at the second depth TH2.

Meanwhile, when the plurality of electrodes 121 perform an injection operation for the third time and the target depth reaches the predetermined third depth TH3, the processor 152 may allow the plurality of electrodes 121 to have a predetermined fourth rest period between application time points of the first high-frequency current of a monopolar type and the second high-frequency current of a bipolar type. Here, the fourth rest period may be a period during which the second high-frequency current of a bipolar type is not applied during a predetermined time. The present disclosure may have the fourth rest period, thereby preventing the occurrence of skin burns in advance and maximizing the optimal skin improvement effect of the monopolar and bipolar types.

At least one component may be added or deleted to correspond to the performance of the components illustrated in FIGS. 1 to 5, 8 to 10, and 12 to 14. Furthermore, it will be easily understood by those skilled in the art that mutual locations of the components may be changed in response to the performance or structure of the system.

FIGS. 6, 7, and 11 illustrate that operations are performed sequentially. However, this is merely illustrative of the technical idea of the present disclosure. Those skilled in the art to which an embodiment of the present disclosure belongs may apply various modifications and variations by changing and performing the order illustrated in FIGS. 6, 7, and 11 or performing one or more operations among a plurality of operations in parallel without departing from the essential characteristics of an embodiment of the present disclosure. The embodiment in FIGS. 6, 7, and 11 is not limited to a time-series order.

Disclosed embodiments are described above with reference to the accompanying drawings. One ordinary skilled in the art to which the present disclosure belongs will understand that the present disclosure may be practiced in forms other than the disclosed embodiments without altering the technical ideas or essential features of the present disclosure. The disclosed embodiments are examples and should not be construed as limited thereto.

What is claimed is:

1. A skin-depth-specific monopolar and bipolar current application apparatus for performing a repetitive needle injection operation, the apparatus comprising:
an electrode unit including a plurality of electrodes of a needle type configured to repeatedly perform an injection operation and a pull operation within a skin;
a drug injection unit configured to inject a drug into the skin;
an energy supply unit configured to selectively apply a first high-frequency current of a monopolar type and a second high-frequency current of a bipolar type to the plurality of electrodes;
a transfer unit configured to move the electrode unit such that the plurality of electrodes reach different target depths within the skin by repeatedly performing the injection operation and the pull operation at a same location; and
a processor configured to:
when the plurality of electrodes respectively reach the different target depths by repeatedly performing the injection operation and the pull operation,
control the energy supply unit such that the first high-frequency current and the second high-frequency current are applied to the plurality of electrodes at current intensities corresponding to the reached different target depths, respectively; and
control the drug injection unit such that the drug is injected into the different target depths.

2. The apparatus of claim 1, wherein the processor is configured to:
when the plurality of electrodes perform the injection operation for a first time, and the target depth is a predetermined first depth, control the energy supply unit such that the first high-frequency current is applied to the plurality of electrodes at a current intensity corresponding to the first depth; and
when the plurality of electrodes perform the injection operation for a second time, and the target depth is a predetermined second depth deeper than the first depth, control the energy supply unit such that the second high-frequency current is applied to the plurality of electrodes at a current intensity corresponding to the second depth.

3. The apparatus of claim 2, wherein the processor is configured to:
when the plurality of electrodes perform the injection operation for a third time, and the target depth is a predetermined third depth deeper than the second depth, control the energy supply unit such that the first high-frequency current is applied to the plurality of electrodes at a current intensity corresponding to the third depth.

4. The apparatus of claim 1, wherein the processor is configured to:
when the plurality of electrodes perform the injection operation for a first time, and the target depth is a predetermined first depth, control the energy supply unit such that the second high-frequency current is applied to the plurality of electrodes at a current intensity corresponding to the first depth; and
when the plurality of electrodes perform the injection operation for a second time, and the target depth is a predetermined second depth deeper than the first depth, control the energy supply unit such that the first high-frequency current is applied to the plurality of electrodes at a current intensity corresponding to the second depth.

5. The apparatus of claim 4, wherein the processor is configured to:
when the plurality of electrodes perform the injection operation for a third time, and the target depth is a predetermined third depth deeper than the second depth, control the energy supply unit such that the second high-frequency current is applied to the plurality of electrodes at a current intensity corresponding to the third depth.

6. A method performed by a skin-depth-specific monopolar and bipolar current application apparatus for performing a repetitive needle injection operation, the method comprising:
moving an electrode unit of the apparatus such that a plurality of electrodes reach different target depths within a skin through a transfer unit of the apparatus by repeatedly performing an injection operation and a pull operation at a same location;
determining, by a processor of the apparatus, whether the plurality of electrodes respectively reach different target depths through repeated performance of the injection operation and the pull operation;
when the plurality of electrodes respectively reach different target depths by repeatedly performing the injection operation and the pull operation, controlling, by the processor, an energy supply unit of the apparatus such that the first high-frequency current and the second high-frequency current are applied to the plurality of electrodes respectively at current intensities respectively corresponding to the reached different target depths; and
controlling a drug injection unit of the apparatus such that the drug is injected into the different target depths.

7. The method of claim 6, wherein the controlling includes:
when the plurality of electrodes perform the injection operation for a first time, and the target depth is a predetermined first depth, controlling, by the processor, the energy supply unit such that the first high-frequency current is applied to the plurality of electrodes at a current intensity corresponding to the first depth; and
when the plurality of electrodes perform the injection operation for a second time, and the target depth is a predetermined second depth deeper than the first depth, controlling, by the processor, the energy supply unit such that the second high-frequency current is applied to the plurality of electrodes at a current intensity corresponding to the second depth.

8. The method of claim 7, wherein the controlling includes:
when the plurality of electrodes perform the injection operation for a third time, and the target depth is a predetermined third depth deeper than the second depth, controlling, by the processor, the energy supply unit such that the first high-frequency current is applied to the plurality of electrodes at a current intensity corresponding to the third depth.

9. The method of claim 6, wherein the controlling includes:
when the plurality of electrodes perform the injection operation for a first time, and the target depth is a predetermined first depth, controlling, by the processor, the energy supply unit such that the second high-frequency current is applied to the plurality of electrodes at a current intensity corresponding to the first depth; and
when the plurality of electrodes perform the injection operation for a second time, and the target depth is a predetermined second depth deeper than the first depth, controlling, by the processor, the energy supply unit such that the first high-frequency current is applied to the plurality of electrodes at a current intensity corresponding to the second depth.

10. The method of claim 9, wherein the controlling includes:
when the plurality of electrodes perform the injection operation for a third time, and the target depth is a predetermined third depth deeper than the second depth, controlling, by the processor, the energy supply unit such that the second high-frequency current is applied to the plurality of electrodes at a current intensity corresponding to the third depth.
